(19) United States
(12) Patent Application Publication
(10) Pub. No.: US 2013/0066239 A1
(43) Pub. Date: Mar. 14, 2013

(54) ULTRASOUND NEUROMODULATION OF THE BRAIN, NERVE ROOTS, AND PERIPHERAL NERVES

(71) Applicant: David J. Mishelevich, Playa del Rey, CA (US)

(72) Inventor: David J. Mishelevich, Playa del Rey, CA (US)

(21) Appl. No.: 13/694,328

(22) Filed: Jan. 16, 2011

Related U.S. Application Data
(60) Provisional application No. 61/325,339, filed on Apr. 18, 2010.

(51) Int. Cl. A61N 7/00

(52) U.S. Cl. CPC A61N 7/00
USPC 601/2

(57) ABSTRACT

Disclosed are methods and systems for non-invasive ultrasound neuromodulation of superficial cortex of the brain or stimulation of nerve roots or peripheral nerves. Such stimulation is used for such purposes as determination of motor threshold, demonstrating whether connectivity to peripheral nerves or motor neurons exists and performing nerve conduction-speed studies. Neuromodulation of the brain allows treatment of conditions such as depression via stimulating superficial neural structures that have connections to deeper involved centers. Imaging is optional.

### CROSS REFERENCE TO RELATED APPLICATIONS

This patent application claims priority to provisional atent pplications Application No. 61/325,339, filed Apr. 18, 2010, entitled “ULTRASOUND NEUROMODULATION OF THE BRAIN, NERVE ROOTS, AND PERIPHERAL NERVES.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for Ultrasound Neuromodulation of the occipital nerve and related neural structures.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. One or a plurality of neural elements can be neuromodulated.

Potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels, which resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

Transcranial Magnetic Stimulation (TMS) has been used for characterization of the motor system. TMS stimulation of the motor cortex is employed to see the motor response in the periphery. The response can be in alternative ways such as Motor Evoked Potentials (MEPs) or measurement of mechanical output. One application is the measurement of conduction time from central to peripheral loci, which can have diagnostic significance. Another is the demonstration of the degree of functional connectivity between the loci. Stimulation more distally such as in the spinal cord nerve roots or the spinal cord itself to measure connectivity from the spinal cord to the periphery. Irrespective of the point of stimulation with the central nervous system, an application is the monitoring of the level of anesthesia present.

While motor-system functions performed using TMS are valuable, they use expensive units, typically costing on the order of $50,000 in 2010 that are large, take a relatively high power, require cooling of the electromagnet stimulation coils, and may be noisy. It would be highly beneficial to be able to perform the same functions using lower-cost stimulation mechanism.

### SUMMARY OF THE INVENTION

It is the purpose of this invention to provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyms through the connections from the superficial cortex to the appropriate deeper centers. Ultrasound can also be used to hit those deeper targets directly. Positron Emission Tomography (PET) or fMRI imaging can be used to detect which areas of the brain are impacted. Compared to Transcranial Magnetic Stimulation, Ultrasound Stimulation systems cost significantly less and do not require significant cooling.

### BRIEF DESCRIPTION OF THE DRAWINGS

FIG. 1 shows ultrasound transducers and EMG sensors at various portions of the nervous system.

FIG. 2 shows a diagram of the ultrasound sensor, ultrasound conduction medium, ultrasound field, and the target.

FIG. 3 shows a block diagram of the control circuit.

### DETAILED DESCRIPTION OF THE INVENTION

It is the purpose of this invention to provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyms through the connections from the superficial cortex to the appropriate deeper centers. Ultrasound can also be used to hit those deeper targets directly. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected.

Ultrasound stimulation can be applied to the motor cortex, spinal nerve roots, and peripheral nerves and generate Motor Evoked Potentials (MEPs). MEPs elicited by central stimulation will show greater variability than those elicited stimulating spinal nerve roots or peripheral nerves. Stimulation results can be recorded using evoked potential or electromyographic (EMG) instrumentation. Muscle Action Potentials (MAPs) can be evaluated without averaging while Nerve Action Potentials (NAPs) may need to be averaged because of the lower amplitude. Such measurements can be used to measure Peripheral Nerve Conduction Velocity (PNCV). Pre-activation of the target muscle by having the patient contract the target muscle can reduce the threshold of stimulation, increase response amplitude, and reduce response latency. Another test is Central Motor Conduction Time (CMCT), which measures the conduction time from the motor cortex to the target muscle. Different muscles are mapped to different nerve routes (e.g., Abductor Digiti Minimi (ADM) represents C8 and Tibialis Anterior (TA) represents L4/5). Still another test is Cortico-Motor Threshold. Cortico-motor excitability can be measured using twin-pulse techniques. Sensory nerves can be stimulated as well and Sensory Evoked Potentials (SEPs) recorded such as stimulation at the wrist (say the median nerve) and recording more peripherally (say over the index finger). Examples of applications include coma evaluation (diagnostic and predictive), epilepsy (measure effects of anti-epileptic drugs), drug effects on cortico-motor excitability for drug monitoring, facial-nerve functionality (including Bell's Palsy), evaluation of dystonia, evaluation of Tourette's Syndrome, exploration of Huntington's Disease abnormalities, monitoring and evaluating motor-neuron diseases such as amyotrophic lateral sclerosis, study of myoclonus, study of postural tremors, monitoring and evaluation of multiple sclerosis, evaluation of movement disorders with abnormalities unrelated to pyramidal-tract lesions, and evaluation of Parkinson's Disease. As evident by the conditions that can be studied with the various functions, neurophysiologic research in a number of areas is supported. Other applications include monitoring in the operating room (say before, during, and after spinal cord surgery). Cortical stimulation can provide relief for conditions such as depression, bipolar disorder, pain, schizophrenia, post-traumatic stress disorder (PTSD), and Tourette syndrome. Another application is stimulation of the phrenic nerve for the evaluation of respiratory muscle function. Clinical neurophysiologic research such as the study of plasticity.

When TMS is applied to the left dorsal lateral prefrontal cortex and depression is treated ‘indirectly” (e.g., at 10 Hz, although other rates such as 1, 5, 15, and 20 Hz have been used successfully as well) due to connections to one or more deeper structures such as the cingulate and the insula as demonstrated by imaging. The same is true for ultrasound stimulation.

A benefit of ultrasound stimulation over Transcranial Magnetic Stimulation is safety in that the sound produced is less with a lower chance of auditory damage. Ironically, TMS produces a clicking sound in the auditory range because of deformation of the electromagnet coils during pulsing, while ultrasound stimulation is significantly above the auditory range.

The acoustic frequency (e.g., typically in that range of 0.3 MHz to 0.8 MHz or above whether cranial bone is to be penetrated or not) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). Power is generally applied at a level less than 60 mW/cm2. Ultrasound pulses may be monophasic or biphasic, the choice made based on the specific patient and condition. Ultrasound stimulators are well known and widely available.

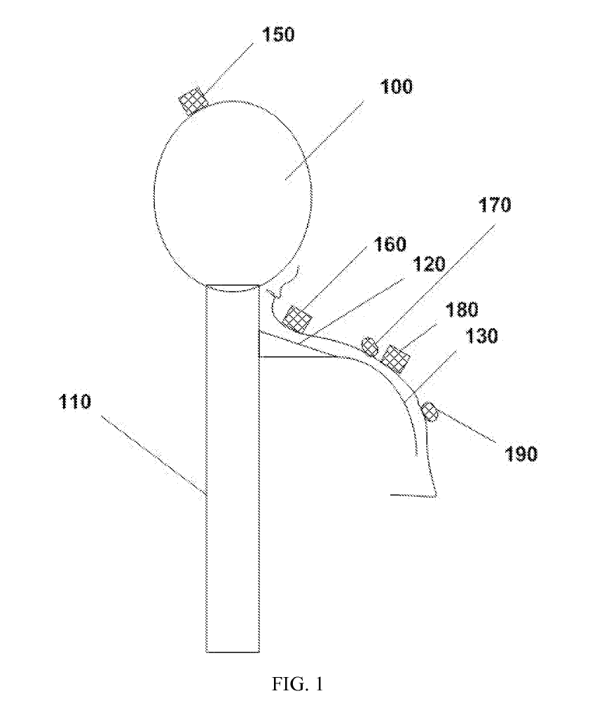

FIG. 1 illustrates placement of ultrasound stimulators EMG and sensors related to head 100, spinal cord 110, nerve root 120, and peripheral nerve 130. Ultrasound transducer 150 is directed at superficial cortex (say motor cortex). For any ultrasound transducer position, ultrasound transmission medium (e.g., silicone oil in a containment pouch) and/or an ultrasonic gel layer. When the ultrasound transducer is pulsed [typically tone burst durations of (but not limited to) 25 to 500 μsec, the conduction time to the sensor at nerve root 170 and/or associated muscles further in the periphery 190. Alternatively ultrasound transducer 160 may be positioned at a nerve root 120 and the conduction time to the electromyography sensor 190 measured. Further, an ultrasound transducer 180 may be positioned over peripheral nerve 130 and the conduction tine to electromyography sensor 190 measured.

Cortical excitability can be measured using single pulses to determine the motor threshold (defined as the lowest intensity that evokes MEPs for one-half of the stimulations. In addition, such single pulses delivered at a level above threshold can be used to study the suppression of voluntarily contracted muscle EMG activity following an induced MEP.

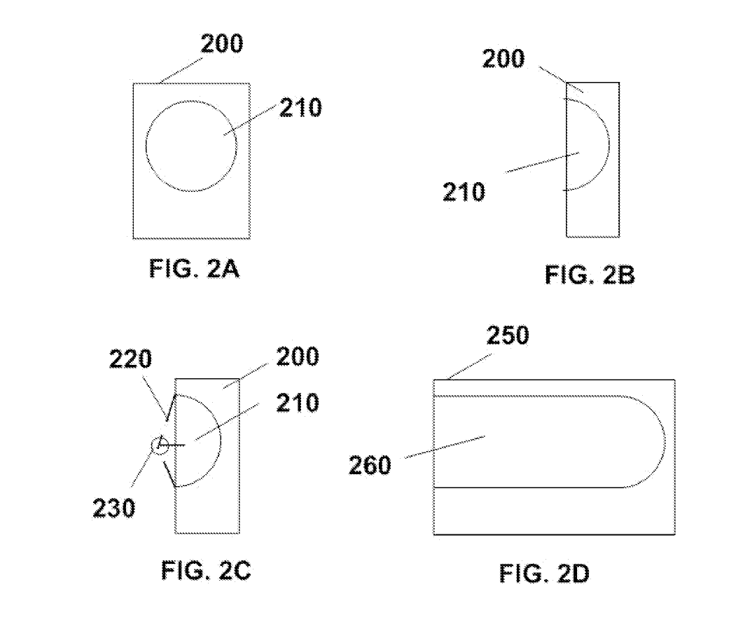

Ultrasound transducer 200 with ultrasound-conduction-medium insert 210 are shown in front view in FIG. 2A and the side view in FIG. 2B. FIG. 2C again shows a side view of ultrasound transducer 200 and ultrasound-conduction-medium insert 210 with ultrasound field 220 focused on the target nerve bundle target 230. Depending on the focal length of the ultrasound field, the length of the ultrasound transducer assembly can be increased with a corresponding increase in the length of ultrasound-conduction-medium insert. For example, FIG. 2D shows a longer ultrasound transducer body 250 and longer ultrasound-conduction-medium insert 260. The focus of ultrasound transducer 200 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Keramos-Etalon can supply a 1-inch diameter ultrasound transducer and a focal length of 2 inches, which with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Other embodiments have mechanisms for focus of the ultrasound including fixed ultrasound array, flat ultrasound array with lens, non-flat ultrasound array with lens, flat ultrasound array with controlled phase and intensity relationships, and ultrasound non-flat array with controlled phase and intensity relationship. Ultrasound conduction medium will be required to fill the space. Examples of sound-conduction media are Dermasol from California Medical Innovations or silicone oil in a containment pouch. If patient sees impact, he or she can move transducer (or ask the operator to do so) in the X-Y direction (Z direction is along the length of transducer holder and could be adjusted as well).

Transducer arrays of the type 200 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” ^{2nd} International Symposium on Therapeutic Ultrasound—Seattle—31/07-Feb. 8, 2002), typically with numbers of ultrasound transducers of 300 or more. Keramos-Etalon in the U.S. is another custom-transducer supplier. The design of the individual array elements and power applied will determine whether the ultrasound is high intensity or low intensity (or medium intensity) and because the ultrasound transducers are custom, any mechanical or electrical changes can be made, if and as required. Blatek in the U.S. also supplies such configurations.

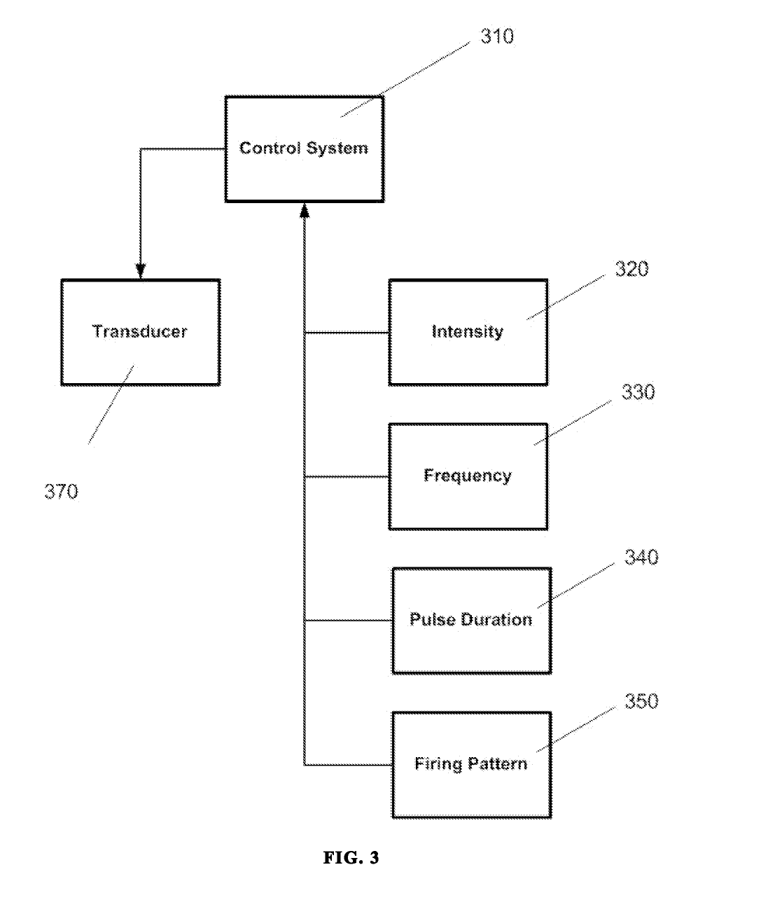

FIG. 3 illustrates the control circuit. Control System 310 receives its input from Intensity setting 320, Frequency setting 330, Pulse-Duration setting 340, and Firing-Pattern setting 350. Control System 310 then provides output to drive Ultrasound Transducer 370 and thus deliver the neuromodulation.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A system of non-invasively neuromodulating the brain using ultrasound stimulation, the system comprising:
aiming an ultrasound transducer at superficial cortex,
applying pulsed power to said ultrasound transducer via a control circuit thereby neuromodulating the target,
whereby results are selected from the group consisting of functional and diagnostic.

2. The system of claim 1, wherein the plurality of control elements is selected from the group consisting of intensity, frequency, pulse duration, and firing pattern.

3. The system of claim 1, wherein the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

4. The system of claim 1 wherein the level ultrasound stimulation is used to assess the excitability of the cortex.

5. A system of non-invasively neuromodulating the brain using ultrasound stimulation, the system comprising:
aiming an ultrasound transducer at a neural target,
applying pulsed power to said ultrasound transducer via a control circuit thereby stimulating the target,
placement of one or a plurality of sensors at a distance from the target, whereby results are selected from the group consisting of diagnostic and monitoring.

6. The system of claim 5, wherein the plurality of control elements is selected from the group consisting of intensity, frequency, pulse duration, and firing pattern.

7. The system of claim 5, wherein the time from stimulation to the time of detection is measured at a sensor where the sensor is placed a location selected from the group consisting of spinal-cord nerve root, peripheral nerve and muscle.

8. The system of claim 5, wherein the system is used for determination of conduction velocity.

9. The system of claim 5, wherein the system is used for monitoring of the level of anesthesia.

10. The system of claim 5, wherein the system is used for monitoring of neural function related to spinal cord surgery.

11. A method of non-invasively neuromodulating the brain using ultrasound stimulation, the method comprising:
aiming an ultrasound transducer at superficial cortex,
applying pulsed power to said ultrasound transducer via a control circuit thereby neuromodulating the target,
whereby results are selected from the group consisting of functional and diagnostic.

12. The method of claim 11, wherein the plurality of control elements is selected from the group consisting of intensity, frequency, pulse duration, and firing pattern.

13. The method of claim 1,1 wherein the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

14. The method of claim 11 wherein the level ultrasound stimulation is used to assess the excitability of the cortex.

15. A method of non-invasively neuromodulating the brain using ultrasound stimulation, the system comprising:
aiming an ultrasound transducer at a neural target,
applying pulsed power to said ultrasound transducer via a control circuit thereby stimulating the target,
placement of one or a plurality of sensors at a distance from the target,
whereby results are selected from the group consisting of diagnostic and monitoring.

16. The method of claim 15, wherein the plurality of control elements is selected from the group consisting of intensity, frequency, pulse duration, and firing pattern.

17. The method of claim 15, wherein the time from stimulation to the time of detection is measured at a sensor where the sensor is placed a location selected from the group consisting of spinal-cord nerve root, peripheral nerve and muscle.

18. The method of claim 15, wherein the system is used for determination of conduction velocity.

19. The method of claim 15, wherein the system is used for monitoring of the level of anesthesia.

20. The method of claim 15, wherein the system is used for monitoring of neural function related to spinal cord surgery.
